(19) United States
(12) Patent Application Publication
(10) Pub. No.: US 2009/0298940 A1
(43) Pub. Date: Dec. 3, 2009

(54) All Natural Antifungal And Antimicrobial Composition And Method Thereof

(76) Inventor: Larry W. Van Etten, Rahramp, NV (US)

Correspondence Address: LAW OFFICE OF TRACY P. JONG, 2775 BUFFALO ROAD, SUITE 3, ROCHESTER, NY, 14624, US

(21) Appl. No.: 12/475,284

(22) Filed: May 29, 2009

Related U.S. Application Data
(60) Provisional application No. 61/057,383, filed on May 30, 2008.

(51) Int. Cl. A61K 31/19; A61P 31/04

(52) U.S. Cl. 514/557

(57) ABSTRACT

In accordance with the present invention, there is provided a novel all natural antifungal composition and method. There is further provided a novel all natural antibiotic and antimicrobial composition and method. There is provided an antifungal and/or antimicrobial composition and method that may be used to treat conditions caused by gram negative bacteria, viruses and fungi, especially molds and mycocytes, with a composition comprising potassium sorbate, lecithin, cellulose, and wax. There is provided a composition and method for administration directly to dermal, oral, gastrointestinal and/or nasal tissues that are affected by molds and mycocytes.

### CROSS REFERENCE TO RELATED APPLICATIONS AND PRIORITY CLAIM

This application claims the benefit of U.S. Ser. No. 61/057,383 filed by Larry VanEtten on May 30, 2008 entitled “All Natural Antifungal and Antimicrobial Composition, Method, and Preparation Thereof.”

### BACKGROUND OF THE INVENTION

1. Field of the Invention

This invention relates to compositions and methods for the treatment of maladies and conditions caused by gram negative bacteria, viral, and/or fungal growth, especially molds, and in particular, all natural antifungal and antimicrobial compositions.

2. Description of Related Art

Many preparations and methods are used to control the growth of harmful or pathogenic biological species. Many are known in the prior art, but recent attention has been focused on compositions that are particularly effective against molds and mycocytes. Molds enter the lungs, contact the skin and enter the nasal passages. Drug treatment to cure mycoses is not optimal. Being eukaryotes, fungi are not amenable to treatment with antibiotics, the most well developed medical treatments available. To treat fungi and mycoses, a variety of drugs interfering with the synthesis of ergosterol or components of the cell wall has been developed; however, they are not successful in many cases. Rather than treat the maladies caused by the molds, it may be possible to simply kill the pathogens by contacting them with an all natural composition immediately after they enter the lungs, contact the skin and enter the nasal passages.

Mold, a member of the fungi family, can cause illness and death, especially in infants, the elderly, diabetics, transplant patients, chemotherapy and cancer patients, or persons that are immuno compromised. Mold can trigger allergic reactions and asthma, memory impairment, migraines, sick building syndrome, dizziness, nosebleeds, or pulmonary hemosiderosis. Current treatments are not fully known or effective. The estimated annual cost to the economy for sick building syndrome (SBS) alone is in the billions of dollars according to the EPA. The liability for mold illnesses and remediation is financially devastating to many industries.

Medical practitioners, veterinarians and patients have continued to seek improved all natural biocides and fungicides that are cost effective to treat conditions caused by a wide variety of pathogenic microorganisms and fungi. Many antifungals and antimicrobials are used in treating animals, humans, birds and marine life. Some are pharmaceutical preparations. Some are dietary supplements. Some are all natural dermatological preparations. However, none is known that has such broad ranging biocidal effects and is so safe and effective it can be used on a wide array of species. Thus, it is desirable to provide compositions and methods that eliminate one or more of the drawbacks and limitations of the prior art.

### SUMMARY OF THE INVENTION

In accordance with this invention, there is provided an improved antifungal and antimicrobial that overcomes the limitations of the prior art by using a unique and previously unknown composition comprising 0.00663 weight percent of positive charge enhancing additive, 0.00663 weight percent of emulsifier, 0.00663 weight percent of carrier, and 0.00663 weight percent of active biocidal component. In a preferred embodiment the novel antifungal and antimicrobial composition further comprises catalyst activated water.

In a preferred embodiment the novel antifungal and antimicrobial composition comprises 0.00663 weight percent of potassium sorbate, 0.00663 weight percent of lecithin, 0.00663 weight percent of cellulose, and 0.00663 weight percent of wax. In a preferred embodiment the novel antifungal and antimicrobial composition further comprises catalyst activated fluorine and chlorine free water.

In accordance with the present invention, there is provided a novel all natural antifungal composition and method. There is further provided a novel all natural antimicrobial composition and method. There is provided an antifungal and/or antimicrobial composition and method that may be used to treat conditions caused by gram negative bacteria, viruses and fungi, especially molds and mycocytes, with a composition comprising potassium sorbate, lecithin, cellulose, and wax. There is provided a composition and method for administration directly to dermal, oral, gastrointestinal and/or nasal tissues that are affected by molds and mycocytes.

It is an object of this invention to provide an all natural antifungal.

It is an object of this invention to provide an all natural antimicrobial.

It is a further object of the present invention to provide an all natural antifungal and/or antimicrobial that may be used to treat conditions caused by gram negative bacteria, viruses and fungi, especially molds.

It is a further object of the present invention to provide an antimicrobial and/or antifungal that is versatile and readily adaptable to use on a wide range of tissues and wide array of species.

It is further an object of the present invention to provide a composition that may be administered directly to the tissues and sites that are affected by molds and mycocytes and/or through which these pathogens are transmitted.

It is a further object of the present invention to provide a method of treating conditions caused by gram negative bacteria, viral, and/or fungal growth, especially molds, with a composition comprising potassium sorbate, lecithin, cellulose, and wax.

It is a further object of the present invention to provide a pharmaceutical preparation and method of elimination and/or inhibiting gram negative bacteria, viral, and/or fungal growth, especially molds, with a composition comprising potassium sorbate, lecithin, cellulose, and wax.

It is a further object of the present invention to provide an all natural topical dermatological preparation and method of elimination and/or inhibiting gram negative bacteria, viral, and/or fungal growth, especially molds on the skin, with a composition comprising potassium sorbate, lecithin, cellulose, and wax.

It is a further object of the present invention to provide an all natural oral rinse and method of elimination and/or inhibiting gram negative bacteria, viral, and/or fungal growth, especially molds, in the oral passages with a composition comprising potassium sorbate, lecithin, cellulose, and wax.

It is a further object of the present invention to provide an all natural nasal rinse and method of elimination and/or inhibiting gram negative bacteria, viral, and/or fungal growth, especially molds, in the nasal passages with a composition comprising potassium sorbate, lecithin, cellulose, and wax.

It is a further object of the present invention to provide an all natural dietary supplement and method of elimination and/or inhibiting gram negative bacteria, viral, and/or fungal growth, especially molds, from the gastrointestinal system with a composition comprising potassium sorbate, lecithin, cellulose, and wax.

Whereas there may be many embodiments of the present invention, each embodiment may meet one or more of the foregoing recited objects in any combination. It is not intended that each embodiment will necessarily meet each objective.

Thus, having broadly outlined the more important features of the present invention in order that the detailed description thereof may be better understood, and that the present contribution to the art may be better appreciated, there are, of course, additional features of the present invention that will be described herein and will form a part of the subject matter of this specification. In this respect, before explaining at least one embodiment of the invention in detail, it is to be understood that the invention is not limited in its application to the details of composition and the arrangements of the process steps set forth in the following description. The present invention is capable of other embodiments and of being practiced and carried out in various ways. Also it is to be understood that the phraseology and terminology employed herein are for the purpose of description and should not be regarded as limiting.

### DETAILED DESCRIPTION OF A PREFERRED EMBODIMENT OF THE INVENTION

The present invention relates to an all natural antifungal and antimicrobial compositions and methods for controlling the growth of pathogenic microorganisms and fungi on humans, aquatic and marine species, birds and animals of all types. The present invention is a novel all natural composition comprising effective amounts of active compounds potassium sorbate, lecithin, cellulose, and wax. The composition may be applied before or after infection by the microbes (e.g. gram negative bacteria, mold, mildew or fungi) as a prophylactic or treatment. Administration may be, for example, via a topical dermatological preparation, via a nasal rinse or via an oral rinse. It may even be administered as a dietary supplement for gastrointestinal exposure.

As used in this specification, antimicrobial means a substance that inhibits one or more microbial life forms, and in particular is effective at inhibiting vegetative disease causing organisms, when introduced in sufficient concentration and duration. As used in this specification, antifungal means a substance that inhibits one or more fungi (including mold) life forms, and in particular is effective at inhibiting vegetative disease causing fungi, molds and mycocytes, when introduced in sufficient concentration and duration.

### Novel Composition

In accordance with this invention, there is provided an improved composition for the treatment of maladies and conditions caused by gram negative bacteria, viral, and fungal growth comprising 0.00663 weight percent of a positive charge enhancing additive, 0.00663 weight percent of a carrier, and 0.00663 weight percent of an active biocidal component wherein a positive charge of the positive charge enhancing additive in combination with the carrier facilitates entry of the active biocidal component into a cellular wall of a fungal spore, virus and gram negative bacteria.

In its most simplistic form, it is the wax and the carrier, the food component cellulose, that make the novel composition effective in treatment and prevention of many maladies (conditions and diseases). The enhancement of the positive charge facilitates better and faster entry into the cellular wall of the pathogens (gram negative bacteria, virus, fungal spore, cancer cell, and the like). When formed as a suspension, an emulsifier is advantageously used to enhance the microdispersion of the wax in the suspension with distilled and/or catalyst activated water, easing both storage (maintaining the dispersion of the solute in the suspension or preventing aggregation of the wax) and use of the product with sprayers and the like. While it is to be understood that the cell death is caused by a combination of the carrier that facilitates entry of the wax as the biocidal component, for ease of description, an embodiment of the novel composition in a suspension form will be described, and will additionally include a positive charge enhancer to attract the cells to the deadly cellulose food source. Through much trial and error, this formulation is the preferred embodiment of the novel composition, but should not be regarded as limiting as such.

In one aspect, the novel composition is in the form of an antibiotic composition effective in treating gram negative bacteria. Preferably, it is in the form of a suspension comprising distilled water and a biocidally effective mixture formed of equal parts of potassium sorbate, lecithin, cellulose, and wax. In a preferred embodiment, the distilled water comprises a solution formed of catalyst activated water and distilled mineralized water wherein the solution contains at least 15% catalyst activated water.

In one aspect, the novel composition is in the form of an antifungal composition effective in treating fungus and yeast conditions. Preferably, it is in the form of a suspension comprising distilled water and a biocidally effective mixture formed of equal parts of potassium sorbate, lecithin, cellulose, and wax. In a preferred embodiment, the distilled water comprises a solution formed of catalyst activated water and distilled mineralized water wherein the solution contains at least 15% catalyst activated water.

In accordance with this invention, there is also provided an improved general antifungal and antimicrobial (viruses, gram negative bacteria, other like pathogens) that overcomes the limitations of the prior art by using a unique and previously unknown composition comprising 0.00663 weight percent of positive charge enhancing additive, 0.00663 weight percent of emulsifier, 0.00663 weight percent of carrier, and 0.00663 weight percent of active biocidal component. In a preferred embodiment the novel antifungal and antimicrobial composition further comprises catalyst activated water (also known as structured water).

In a preferred embodiment the novel antifungal and antimicrobial composition comprises 0.00663 weight percent of potassium sorbate as the positive charge enhancing additive, 0.00663 weight percent of lecithin as the emulsifier, 0.00663 weight percent of cellulose as the carrier, and 0.00663 weight percent of wax as the biocidal component. In a preferred embodiment the novel antifungal and antimicrobial composition further comprises catalyst activated fluorine and chlorine free water. In another embodiment, catalyst activated water in gel format is used.

The present invention relates to active compounds comprising a mixture of equal parts of potassium sorbate, lecithin, cellulose, and wax to form one or more biocides (e.g. antifungal and antimicrobials). The active compounds are employed in a purity of from about 80% to about 100%, preferably from about 95% to 100%. In a preferred embodiment of the novel antifungal and antimicrobial composition, a suspension comprises from about 0.245 to about 0.26 grams, and preferably 0.25 grams, of potassium sorbate; from about 0.245 to about 0.26 grams, and preferably 0.25 grams, of lecithin; from about 0.245 to about 0.26 grams, and preferably 0.25 grams, of cellulose; and from about 0.245 to about 0.26 grams, and preferably 0.25 grams, of wax per gallon of solution. The active compounds are in a ratio of from about 0.0002652:1 (active compounds: water) with water. Preferably, the water comprises a solution of 15% catalyst activated water such as chlorine and fluorine free mineralized water and 85% distilled mineralized water. Most preferably, catalyst activated water such as Willard Water from South Dakota is used with the composition of the present invention. Most preferably, cactus wax is used with the composition of the present invention. Most preferably, soy lecithin is used with the composition of the present invention.

In one preferred embodiment, the boiling point of the composition is 100 degrees Celsius and the specific gravity is 1.2 (water=1). The composition is fully soluble in water, is odorless and appears as an opaque liquid suspension.

The active compounds are preferably mixed with catalyst activated water (CAW) such that the weight ratio of potassium sorbate to CAW is 0.0441% or 0.000441:1 (potassium sorbate:CAW); the weight ratio of lecithin to CAW is 0.0441% or 0.000441:1 (lecithin:CAW); the weight ratio of cellulose to CAW is 0.0441% or 0.000441:1 (cellulose:CAW); and the weight ratio of wax to CAW is 0.0441% or 0.000441:1 (wax:CAW) (weights at 68 degrees Fahrenheit). This concentrated suspension can be diluted with distilled mineralized water to form a total of one gallon of product.

There is also provided a method of treatment for conditions caused by gram negative bacteria, viral, and/or fungal growth, especially molds and mycocytes, with a composition comprising an antifungal and/or antimicrobial effective amount (e.g. therapeutic dosage) of the active compounds of potassium sorbate, lecithin, cellulose, and wax. In one embodiment, the effective amount of each active compound is in a 1:1:1:1 ratio or equal parts. In a preferred embodiment of the method, the composition comprises 0.00663 weight percent of potassium sorbate, 0.00663 weight percent of lecithin, 0.00663 weight percent of cellulose, and 0.00663 weight percent of wax. In one aspect, the composition is a suspension prepared according to the novel process of the present invention.

Use of the term “antimcrobial effective amount” (therapeutic dosage) herein means an amount sufficient to prevent, reduce, or cease the growth of microbes and their harmful effects substantially equally to or better than other known anticrobial compositions. The actual comparable amount varies depending on the traditional antimcrobial being replaced and the microbe being protected against. With respect to “antibacterial effective amount (therapeutic dosage) as used herein the same definition applies for anticrobial effective amount except it apples only to gram negative bacteria. Use of the term antifungal effective amount”(therapeutic dosage) herein means an amount sufficient to prevent, reduce, or cease the growth of fungi, molds and/or mycocyte, and their harmful effects substantially equally to or better then other known antifungal compositions. The actual comparable amount varies depending on the traditional antifungal being replaced and the fungi being protected against.

The novel composition acts as an antifungal and antimicrobial composition by using wax as the biocidal component to encapsulate the microorganism, thereby effectively suffocating the fungal spore, virus or microbe. Gram-negative bacteria, mold, fungus, and viruses share the common biological characteristic of being attracted to a positive charge, and in particular, a positive charge of the ionic potassium sorbate. By combining the biocidally effective components with the food components of the microbes/fungi, the biocide can be ingested in an amount effective to act as a biocide or biostat.

The novel composition is also effective against other pathogens such as cancers. Administration to several cancer patients has shown the treatment to be effective at reducing or eliminating cancer such as esophageal cancer.

Molds require one or more organic nutrients. Because molds must absorb or transport their nutrients through the cell surface, they compete with bacteria for organic nutrients. Many molds have an advantage over the bacteria in this competition, because they can secrete digestive extra cellular enzymes such as cellulases. Thus they can degrade an otherwise insoluble organic substrate into its smaller soluble subunits, which they then absorb and use as sources of carbon and energy. This enables the molds to use carbon sources, including cellulose, that are unavailable to most other microorganisms. Thus, the use of cellulose as a carrier is advantageous for the treatment of molds in particular.

The novel biocidal (antifungal and antimicrobial) composition attaches to the effected surfaces of gram negative bacteria, fungi, mold or virus and kill it. The charge of the potassium sorbate attracts the gram negative bacteria, fungi, mold or virus and facilitates its entry into the digestive canals quickly. The novel antifungal and antimicrobial composition effectively suffocates the microorganism by blocking the air/digestive action pathways. Gram-negative bacteria can not burp, flactuate, or otherwise clear its own digestive canals. Since the composition acts much more quickly than the microorganism can naturally digest, and it blocks the air/digestive action pathways, and thus immediately renders the microorganism a lethal dose. The effectiveness of the composition is in part due to its active mechanism's natural harmony with the workings of the microorganisms. Specifically, the positively charged potassium sorbate is attracted to the opposing charge of the microorganism permitting entry of the biocidal components into the microorganism's cellular wall, and thereafter, the wax blocks the pathway and encapsulates the spore/microbe.

The active compound potassium sorbate functions as a charge enhancing additive, specifically a positive charge. Potassium sorbate is also known as 2,4-hexadienoic acid potassium salt {potassium sorbate [K(CH₃CH═CHCH═CHCO₂)]. The molecular formula of potassium sorbate is C₆H₇O₂K and its systematic name is potassium (E,E)-hexa-2,4-dienoate. It has a molecular weight of 150.22 g/mol. It is very soluble in water (58.2% at 20° C.). It is prepared by the reaction of sorbic acid with potassium hydroxide.

While potassium sorbate has been known for its antimicrobial and antifungal properties for food and cosmetics preservatives, when used in accordance to the present invention, it has an unexpected, intriguing nature on account of its ionic properties. The positively charged potassium sorbate seeks out the opposite charge of the microorganism and draws the gram negative bacteria, fungi, mold or virus to it, or conversely, is drawn to the gram negative bacteria, fungi, mold or virus. The natural antimicrobial and antifungal properties of potassium sorbate enhance the overall efficacy of the composition.

Potassium sorbate is considered to be safe because of its long term safety record and non-toxic profile. Potassium sorbate is non-irritating and non-sensitizing. Allergic reactions are rare and it is well tolerated when administered internally. Thus, it is suitably used for topical, oral and nasal preparations and methods in accordance with the present invention.

The active compound lecithin functions as a natural emulsifier that allows penetration of the potassium sorbate. It may also function as a surfactant and dispersant. Preferably, lecithin is extracted from soybean plants (SID: 48414465). Other forms of lecithin tend to form a ‘glob’ even when the temperature during mixing is held at 100 degrees Celsius for an extended period of time (more than 30 seconds).

The active compound cellulose functions as a carrier of the charged material and wax. Once charged, the cellulose will not alter the potassium sorbate or its positive charge. It assists in holding the charged material at its charge state during delivery to the microorganism.

The active compound wax functions to encapsulate and suffocate the gram negative bacteria, fungi, mold or virus spore or molecule. Preferably, the wax comprises a western wax from cactus. New Mexico wax has been the best for the following characteristics: melting, delivery, encapsulation, blending, mixing, non-coagulating in the container and body/surface, and general efficacy.

In another embodiment, the wax comprises beeswax. Beeswax, like honey, contains one or more sylfhydrils which are natural antibodies. Beeswax also contains one or more capsaicin (capsinoids) compounds, some of which are natural antimicrobials. These enhance the biocidal properties of the composition.

The active substance compositions thus obtainable can be used directly as such or after diluting. In addition, the compositions according to the present invention can also comprise conventional additives such as, for example, viscosity-modifying additives, anti-foam agents, anti-freeze agents, dispersants, emulsifiers, surfactants, binders, waxes and the like. It is important that the charge of the composition is not modified.

The composition of the present invention can be formulated in the form of directly sprayable solutions, powders and suspensions or in the form of highly concentrated aqueous, oily or other suspensions, dispersions, emulsions, oil dispersions, pastes, dusts, materials for broadcasting or granules, and applied for spraying, atomizing, dusting, broadcasting or watering. The form depends on the intended purpose. In any case, it should as fine and uniform as possible a distribution of the mixture according to the present invention. The formulations are prepared in a manner known in the art, such as, for example, using appropriate carriers and/or solvents, and inert additives such as emulsifiers, surfactants, dispersants and the like.

In a preferred method of use, the composition is prepared as a suspension according to the novel process described herein and is sprayed onto the affected site and allowed to penetrate for three to five seconds.

### A Novel Method of Treatment

Treating maladies and conditions caused by gram negative bacteria, viral, and/or fungal growth is accomplished by topically contacting an affected surface of a subject's body with a composition comprising a suspension of distilled water and a biocidally effective mixture formed of equal parts a positive charge enhancing additive, an emulsifier, a carrier, and an active biocidal component; and allowing the composition to penetrate the affected surface for three to five seconds. As described above, in a preferred formulation, the biocidally effective mixture comprises equal parts of potassium sorbate, lecithin, cellulose and wax.

The subject may be humans, aquatic and marine species, birds and animals of all types or even a plant species.

There is also provided a method of inhibiting the growth of fungi and pathogenic microbes in a subject by enteral administration of a therapeutically effective dosage of a composition having cellulose as a carrier for a wax to be ingested by the pathogen such that the ingested wax encapsulates the pathogen, thereby effectively suffocating the pathogen.

There is also provided a method of inhibiting the growth of pathogens in a subject that begins with combining a biocidally effective mixture containing a positive charge and a wax with a food component of the pathogen to form a therapeutic composition (the pathogen has a negative charge) followed by administration of a therapeutic dosage of the therapeutic composition to the subject, causing the pathogen to be attracted to the positive charge and thereby causing the pathogen to ingest the biocidally effective mixture. The positive charge in combination with the food component facilitates entry of the biocidally effective mixture into a digestive canal of the pathogen, thereby effectively suffocating the pathogen when the wax blocks an air or digestive action pathway of the pathogen.

### Treatment of Fungi, Molds and Mycocytes

The compositions can be topically applied to the skin to protect the skin from fungi, molds, mycocytes and other microbes, and to treat or retard their growth which increases the likelihood of the onset of unhealthy conditions, maladies and diseases. The compositions can also be applied to the nasal passages as a nasal rinse, aerosol or spray. The compositions can be topically applied to the oral passages as an oral rinse (swish) or spray. The compositions can be ingested as dietary supplements to address gastrointestinal exposures.

One beneficial application of these novel compositions and methods is for workers or residents exposed to environmental molds and pathogens. Exposure routes generally include skin, inhalation, and ingestion. An application before and/or after exposure may prevent the pathogens from harmful effects it they were killed directly at the site of exposure. For example, a preventative or disinfecting effect is realized by rinsing the nasal passages to rid of inhaled pathogens present in the nose, rinsing the oral passages for ingested pathogens present in the mouth or throat, or topical application to the skin for pathogens that are present on the skin surface from dermal contact. Preferably this is administered both before and after exposure.

The composition is effective against the following molds and/or the mycotoxins they produce: Aspergillus, Cladosporium, Penicillium, Stachybotrys, and Trichoderma. The composition is effective against the following fungi: Paracoccidioides brasiliensis, Candida albicans, Histoplasma, Cryptococcus, B. dermatitidis, Coccidioides immitis, and Paracoccidioides brasiliensis. Exposure routes include skin, inhalation, and ingestion.

Thus, there is provided a novel all natural antifungal composition and method. There is further provided a novel all natural antimicrobial composition and method. There is provided an antifungal and/or antimicrobial composition and method that may be used to treat conditions caused by gram negative bacteria, viruses and fungi, especially molds and mycocytes, with a composition comprising potassium sorbate, lecithin, cellulose, and wax. There is provided a composition and method for administration directly to dermal, oral, gastrointestinal and/or nasal tissues that are affected by molds and mycocytes.

For a more complete understanding of the potential health benefits that the novel compositions and methods may provide, we will briefly discuss some pathogenic molds and related health conditions. These are for illustrative purposed and it is intended that the present invention may be used for treatment/prevention of conditions related to these and other fungi, molds, viruses and gram negative bacteria. It may also be used for other pathogens such as cancers.

Aspergillus is a genus of mold which can be found within indoor environments and of which certain species are pathogenic. Pathogenic species of Aspergillus fungus cause an infection, a growth, or an allergic response. Aspergillus versicolor Conidia is commonly found in soil, hay, cotton and dairy products, may be of various colors, and displays great variability in colony pattern and size. Certain pathogenic species are agents of opportunistic infections in debilitated patients. A. versicolor can produce a mycotoxin sterigmatocystin and cyclopiaxonic acid. These toxins can cause gastrointenstinal disruption such as diarrhea or upset stomach, inflammatory diseases in lungs and other organs, fungus balls (mycetoma) to form in the lungs, infection of the anterior and posterior chambers of the eye (and can lead to blindness), clinical infections in persons who have weak immune systems, and have been reported to be a kidney and liver carcinogen. Disseminated aspergillosis is a rare, acute and usually fatal infection that produces Septicemia, Thrombosis, and can affect virtually any organ, but especially the heart, lungs, brain, and kidneys.

Cladosporium is a genus of mold which can be found airborne within indoor environments and is the most common of the so-called black molds (because it produces a black pigment that protects it from ultraviolet light). Certain pathogenic species cause severe infections when it comes in contact with small cuts or abrasions on the skin, cause allergic reactions and other restrictive airway conditions. Prolonged exposure can weaken the immune system allowing opportunistic bacteria and viruses to infect the host. Cladosporium may be linked to some cases of impotence. Volatile Organic Compounds (VOC's) are attributed to this mold at certain stages of its existence. It produces no major mycotoxins of concern.

Penicillium is a genus of mold which can be found within indoor environments. Certain pathogenic species debilitate the immune system of immunosuppressed persons with secondary infections. Volatile Organic Compounds (VOC's) are attributed to this mold at certain stages of its existence.

Stachybotrys is a greenish-black fungus found worldwide that colonizes particularly well in high-cellulose material, such as straw, hay, paper, dust, lint, and cellulose-containing building material such as fiber board, and gypsum board that becomes chronically moist or water damage due to excessive humidity, water leaks, condensation or flooding. Chronic exposure to the toxin produced by this fungus causes cold and flu symptoms, sore throats, diarrhea, headaches, fatigue, dermatitis, paralyzes sperm at low doses and may cause lung bleeding infants and immunosupressed individuals.

Mycotoxins are secondary metabolites of molds that exert toxic effects on animals and humans. The toxic effect of mycotoxins on animal and human health is referred to as mycotoxicosis, the severity of which depends on the toxicity of the mycotoxin, the extent of exposure, age and nutritional status of the individual and possible synergistic effects of other chemicals to which the individual is exposed. Known mycotoxins all seem to work by disrupting cell function in animals and humans, interfering with vital cell processes such as protein, RNA, and DNA synthesis.

The pathogenic fungus Candida albicans is responsible for most of the fungal infections that occur in immuno-compromised individuals. Candida species are the fourth-leading cause of nosocomial infections, and in those patients with candidemia, the attributable mortality rate is 35%. Furthermore, oropharyngal candidiasis occurs in approximately 70% of patients with AIDS, ca. 70% of all women experience at least one episode of vaginitis caused by Candida and 20% will experience recurrent disease.

Histoplasma fungus is enters macrophages or trachea epithelial cells, and causes pneumonia similar to tuberculosis and affects the renal cortex and nervous system.

By inhalation, Cryptococcus causes a chronic, subacute to acute pulmonary, systemic or meningitic disease. Infection suppresses the immune response and gives a poor inflammatory response and induces a proliferative response in human peripheral blood mononuclear cells thereby enhancing HIV replication.

B. dermatitidis causes “Chicago disease’. Primary infection occurs in the lungs, causes an influenza-like pneumonia, later affecting bones and skin.

Coccidioides immitis causes “Valley fever’ that can become acute, chronic, severe or fatal and is manifest in lung, bone and joints, or may disseminate to meningitis.

Paracoccidioides brasiliensis causes a granulomatous disease that originates as a pulmonary infection and disseminates to result in ulcerative granulomatoma in the nasal and buccal, occasionally in the gastrointestinal mucosa or lymph nodes.

Novel Process for Manufacturing a Novel Antifungal and/or Antimicrobial Composition

Preferably, the composition of the present invention is prepared by a process comprising the steps of:

Preparing a solution of 15% catalyst activated water and distilled mineralized water;

Heating the solution to about 100 degrees Celsius;

Adding 0.25 grams wax;

Mixing the solution at a speed of from about 1000 rpm to about 1250 rpm, preferably from about 1150 rpm to about 1250 rpm, while maintaining a temperature of from about 80 to about 120 degrees Celsius, preferably from about 95 to about 100 degrees Celsius; for a period of from about 30 to about 60 seconds;

Adding 0.25 grams potassium sorbate;

Mixing the solution at a speed of from about 1000 rpm to about 1250 rpm, preferably from about 1150 rpm to about 1250 rpm, while maintaining a temperature of from about 80 to about 120 degrees Celsius, preferably from about 95 to about 100 degrees Celsius; for a period of from about 30 to about 60 seconds;

Adding 0.25 grams cellulose;

Mixing the solution at a speed of from about 1000 rpm to about 1250 rpm preferably from about 1150 rpm to about 1250 rpm while maintaining a temperature of from about 80 to about 120 degrees Celsius preferably from about 95 to about 100 degrees Celsius; for a period of from about 5 to about 60 seconds;

Adding 0.25 grams lecithin;

Mixing the solution at a speed of from about 1000 rpm to about 1250 rpm preferably from about 1150 rpm to about 1250 rpm while maintaining a temperature of from about 80 to about 120 degrees Celsius preferably from about 95 to about 100 degrees Celsius; for a period of from about 5 to about 60 seconds to form a suspension;

Filtering the suspension by a filter greater than 5 microns; and

cooling the suspension to ambient temperature.

The suspension may be made in greater or smaller quantities by a batch process with appropriate adjustments to the ingredients to maintain the desired ratios directly proportionate to the batch size.

Preferably, the equipment used throughout the mixing and blending process is formed of materials that will not carry an electronic charge or current of any kind on the surface of the vessels or with its materials. It is important that during the process, no charge, irrespective of its significance in size or polarity, may be added or taken from the batch.

### Storage, Spoliation and Recharging

Preferably, the antifungal and/or antimicrobial composition is stored in bottle or container formed of a material that will not carry an electronic charge or current of any kind on the surface of the vessels or with its materials. Preferably, the lid is sealed at all times except during use, and especially during a storm to prevent changes in polarity or charge of the composition.

Preferably, the composition is not frozen and measures to prevent freezing are undertaken.

Should the composition lose its charge and effectively become spoiled against its antifungal and/or antimicrobial properties, it can be recharged.

### All Natural Preparations: Oral Rinse, Nasal Rinse and Dermatological Preparation

In accordance with the present invention, there is provided an all natural topical preparation and method of elimination and/or inhibiting gram negative bacteria, viral, and/or fungal growth, especially molds, with a composition comprising effective (antifungal and/or antimicrobial) amounts of potassium sorbate, lecithin, cellulose, and wax. Preferably, a suspension prepared according to the novel process described herein is used in this application.

It is a further object of the present invention to provide an all natural oral rinse and method of elimination and/or inhibiting gram negative bacteria, viral, and/or fungal growth, especially molds, with a composition comprising effective (antifungal and/or antimicrobial) amounts of potassium sorbate, lecithin, cellulose, and wax. Preferably, a suspension prepared according to the novel process described herein is used in this application.

It is a further object of the present invention to provide an all natural nasal rinse and method of elimination and/or inhibiting gram negative bacteria, viral, and/or fungal growth, especially molds, with a composition comprising effective (antifungal and/or antimicrobial) amounts of potassium sorbate, lecithin, cellulose, and wax. Preferably, a suspension prepared according to the novel process described herein is used in this application.

### Other Pharmaceutical Preparations

In accordance with the present invention, there is provided a pharmaceutical preparation and method of elimination and/or inhibiting gram negative bacteria, viral, and/or fungal growth, especially molds, with a composition comprising potassium sorbate, lecithin, cellulose, and wax. In one aspect of the present invention, pharmaceutical preparations may be prepared for treatment of humans and other animals, birds, marine life and living species. Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein. The amount of composition administered will be dependent upon the condition being treated, the subject being treated, on the subject's weight, the severity of the affliction, the manner of administration and the judgment of the personalizing physician or veterinarian.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipient and auxiliaries which facilitate processing of the compositions compounds into preparation which can be used pharmaceutically. Proper formulation is dependent upon the route of administration chosen.

For injection, the agents of the invention may be formulated in aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, the compositions can be formulated readily by combining the active compositions with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP).

If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as fit, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a power mix of the compound and a suitable powder base such as lactose or starch.

The compositions may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active composition may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compositions may also be formulated in rectal compositions such as suppositories or retention enemas, e.g., containing conventional suppository bases such as cocoa butter or other glycerides.

In addition to the formulations described previously, the compositions may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable routes of administration may, for example, include oral, rectal, transmucosal, transdermal, or intestinal administration, parenteral delivery, including intramuscular, subcutaneous, intramedullary injections, as well as intrathecal, direct intraventricular, intravenous, intraperitoneal, intranasal, or intraocular injections.

Alternatively, one may administer the composition in a local rather than systemic manner, for example, via injection of the compound directly into an affected area, often in a depot or sustained release formulation.

Furthermore, one may administer the drug in a targeted drug delivery system, for example, in a liposome coated with an antibody specific for affected cells. The liposomes will be targeted to and taken up selectively by the cells.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a composition of the invention formulated in a compatible pharmaceutical carrier may also be prepared, placed in an appropriate container, and labeled for treatment of an indicated condition. Suitable conditions indicated on the label may include treatment of a disease.

By way of illustration, a composition according to the present invention may be used for the treatment and prevention of legionella, botulism, norovirus, h5n1, artillery fungus, yeast infections, Stachybotrys chartarum, Aspergillus niger, Chaetomium globosum, Staphylococcus aureus, Penicillium variabile Trichophyton mentagrophytes (Athlete's Foot fungus), Mycobacterium bovis BCG (Tuberculosis) Streptococcus salivarius, Streptococcus pyogenes, Salmonella choleraesuis, Pseudomonas aeruginosa, Escherichia coli (E. coli), Pellicularia filamentosa, Herpes simplex types 1/F and 2/G (oral, ocular and genital), Polio virus type 1, Influenza A₂ (Japan 305/57 Asian Strain), HIV-1 (AIDS virus), Vaccinia virus, Canine parvovirus, Cytomegalovirus, Coronavirus (Infectious bronchitis virus), Methicillin resistant Staphylococcus aureus (MRSA), and Vancomycin resistant Enterococcus feacium (VRE). A composition according to the present invention may be used as agents for treating immunodeficiency disorders, cancer, wound healing and infectious diseases.

A composition according to the present invention may be used as agents for treating digestive system disturbances including diarrhea, nausea, vomiting, intestinal hemorrhage, liver effects; respiratory system disturbances including coughing, shortness of breath, bleeding from lungs or nose; nervous system disturbances including tremors, problems with coordination; depression, severe fatigue, headache, inability to concentrate, memory problems; Immune system disturbances including suppression resulting in frequent or unexplained illnesses, frequent cold and flu-like symptoms; and skin disturbances including rash, burning or “crawling” sensation, itching, and red patches that do not respond to antibiotics.

A composition according to the present invention may be used as agents for treating diseases caused by mycotoxins including ochratoxins, fumonisins, and zearalenone.

A composition according to the present invention may be used as agents for treating ergotism.

A composition according to the present invention may be used as agents for treating diseases and cancers, especially of the liver, caused by immunosuppressive, mutagenic, teratogenic and carcinogenic aflatoxins.

A composition according to the present invention may be used as agents for treating conditions and diseases caused by aflatoxins such including encephalopathy and fatty degeneration of viscera, fatty liver and kidneys, jaundice, kwashiorkor and marasmic kwashiorkor, a form of acute food-poisoning called “moldy sugarcane poisoning” and severe cerebral oedema.

A composition according to the present invention may be used as agents for treating conditions and diseases caused by Ochratoxins including etiology, endemic nephropathy and urothelial tumors.

A composition according to the present invention may be used as agents for treating conditions and diseases caused by deoxynivalenol (DON), also known as vomitoxin, nivalenol (NIV), diacetoxyscirpenol (DAS), and T-2 toxin, including depression of immune responses, nausea, vomiting, trichothecene mycotoxicosis, and alimentary toxic aleukia.

A composition according to the present invention may be used as agents for treating conditions and diseases caused by Zearalenone (previously known as F-2) including infertility, vulval oedema, vaginal prolapse and mammary hypertrophy in females and feminization of males, atrophy of testes, enlargement of mammary glands, and “scabby grain toxicosis”.

A composition according to the present invention may be used as agents for treating conditions and diseases caused by mycotoxins produced by Fusarium moniliforme and related species.

A composition according to the present invention may be used as agents for treating conditions and diseases caused by Aspergillus, sterigmatocystin and cyclopiaxonic acid including gastrointenstinal disruption, diarrhea, upset stomach, inflammatory diseases in lungs and other organs, fungus balls (mycetoma), infection of the anterior and posterior chambers of the eye, kidney cancer, liver cancer, Septicemia, and Thrombosis.

A composition according to the present invention may be used as agents for treating conditions and diseases caused by Cladosporium skin/wound infections, allergic reactions, restrictive airway conditions, weakened immune system, and impotence.

A composition according to the present invention may be used as agents for treating conditions and diseases caused by Penicillium.

A composition according to the present invention may be used as agents for treating conditions and diseases caused by Stachybotrys including cold and flu symptoms, sore throats, diarrhea, headaches, fatigue, dermatitis, fertility disorders, and lung bleeding infants and immunosupressed individuals.

A composition according to the present invention may be used as agents for treating conditions and diseases caused by Candida albicans including nosocomial infections, oropharyngal candidiasis, and vaginitis.

A composition according to the present invention may be used as agents for treating conditions and diseases caused by Histoplasma including pneumonia, renal cortex disorders and nervous system disorders.

A composition according to the present invention may be used as agents for treating conditions and diseases caused by Cryptococcus including chronic, subacute to acute pulmonary, systemic or meningitic diseases, immunosuppressive disorders and HIV.

A composition according to the present invention may be used as agents for treating conditions and diseases caused by B. dermatitidis including “Chicago disease’.

A composition according to the present invention may be used as agents for treating conditions and diseases caused by Coccidioides immitis including “Valley fever’ and meningitis.

A composition according to the present invention may be used as agents for treating conditions and diseases caused by Paracoccidioides brasiliensis including pulmonary infections, ulcerative granulomatoma in the nasal and buccal, and gastrointestinal mucosa or lymph nodes.

### Dietary Supplements

Dietary supplements suitable for use in the present invention include compositions wherein the active ingredients are contained in an effective amount to achieve its intended purpose. In one aspect, the intended purpose is to kill mold and fungi resulting from gastrointestinal exposure. More specifically, an effective amount means an amount effective to prevent development of or to alleviate the existing symptoms of the subject being treated. Determination of the effective amounts is well within the capability of those skilled in the art, especially in light of the detailed disclosure provided herein. The amount of composition administered will be dependent upon the condition being treated, the subject being treated, on the subject's weight, the severity of the affliction, the manner of administration and the judgment of the personalizing physician or veterinarian.

The ingredients of the dietary supplement of this invention are contained in acceptable excipients and/or carriers for oral consumption. The actual form of the carrier, and thus, the dietary supplement itself, may not be critical. The carrier may be a liquid, gel, gel cap, capsule, powder, solid tablet (coated or non-coated), tea or the like. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, agar, lactose, methylcellulose, povidone, carboxymethylcellulose, corn starch, and the like (including mixtures thereof). The various ingredients and the excipient and/or carrier are mixed and formed into the desired form using conventional techniques. Dose levels/unit can be adjusted to provide the recommended levels of ingredients per day in a reasonable number of units.

The dietary supplement may also contain optional ingredients including, for example, herbs, vitamins, minerals, enhancers, colorants, sweeteners, flavorants, inert ingredients, and the like. Such optional ingredients may be either naturally occurring or concentrated forms. Selection of one or several of these ingredients is a matter of formulation, design, consumer preference and end-user. The amounts of these ingredients added to the dietary supplements of this invention are readily known to the skilled artisan. Guidance to such amounts can be provided by the U.S. RDA doses for children and adults.

I claim:

1. A composition for the treatment of maladies and conditions caused by gram negative bacteria, viral, and fungal growth comprising 0.00663 weight percent of a positive charge enhancing additive, 0.00663 weight percent of a carrier, and 0.00663 weight percent of an active biocidal component wherein a positive charge of the positive charge enhancing additive in combination with the carrier facilitates entry of the active biocidal component into a cellular wall of a fungal spore, virus and gram negative bacteria.

2. The composition of claim 1 wherein the positive charge enhancing additive is potassium sorbate.

3. The composition of claim 1 wherein the composition is a suspension and further comprises an emulsifier.

4. The composition of claim 1 wherein the carrier is cellulose.

5. The composition of claim 1 wherein the active biocidal component is wax.

6. The composition of claim 1 wherein the composition is a suspension and further comprises catalyst activated water.

7. An antifungal composition in the form of a suspension comprising distilled water and a biocidally effective mixture formed of equal parts of potassium sorbate, lecithin, cellulose and wax.

8. The antifungal composition of claim 7 wherein the distilled water comprises a solution formed of catalyst activated water and distilled mineralized water wherein the solution contains at least 15% catalyst activated water.

9. The antifungal composition of claim 8 wherein the biocidally effective mixture is mixed with catalyst activated water (CAW) such that the weight ratio of potassium sorbate to CAW is 0.0441%; the weight ratio of lecithin to CAW is 0.0441%; the weight ratio of cellulose to CAW is 0.0441%; and the weight ratio of wax to CAW is 0.0441%.

10. An antibiotic composition effective in treating gram negative bacteria in the form of a suspension comprising distilled water and a biocidally effective mixture formed of equal parts of potassium sorbate, lecithin, cellulose, and wax.

11. The antibiotic composition of claim 10 wherein the distilled water comprises a solution formed of catalyst activated water and distilled mineralized water wherein the solution contains at least 15% catalyst activated water.

12. The antibiotic composition of claim 11 wherein the biocidally effective mixture is mixed with catalyst activated water (CAW) such that the weight ratio of potassium sorbate to CAW is 0.0441%; the weight ratio of lecithin to CAW is 0.0441%; the weight ratio of cellulose to CAW is 0.0441%; and the weight ratio of wax to CAW is 0.0441%.

13. An antimicrobial composition for the treatment of maladies and conditions caused by gram negative bacteria, viral, and/or fungal growth mold inhibiting comprising a suspension formed of cellulose, distilled water and microdispersed wax.

14. The composition of claim 13 wherein the composition further comprises a positive charge enhancing additive.

15. The composition of claim 14 wherein the positive charge enhancing additive is potassium sorbate.

16. The composition of claim 13 wherein the composition further comprises an emulsifier.

17. A method for treating maladies and conditions caused by gram negative bacteria, viral, and fungal growth, comprising topically contacting an affected surface of a subject's body with a composition comprising a suspension of distilled water and a biocidally effective mixture formed of equal parts a positive charge enhancing additive, an emulsifier, a carrier, and an active biocidal component; and allowing the composition to penetrate the affected surface for three to five seconds.

18. The method of claim 17 wherein the biocidally effective mixture comprises equal parts of potassium sorbate, lecithin, cellulose and wax.

19. A method of inhibiting the growth of fungi and pathogenic microbes in a subject comprising enteral administration of a therapeutically effective dosage of a composition having cellulose as a carrier for a wax to be ingested by the pathogen such that the ingested wax encapsulates the pathogen, thereby effectively suffocating the pathogen.

20. A method of inhibiting the growth of pathogens in a subject, comprising the steps of:
combining a biocidally effective mixture containing a positive charge and a wax with a food component of the pathogen to form a therapeutic composition, wherein the pathogen has a negative charge;
administration of a therapeutic dosage of the therapeutic composition to the subject, causing the pathogen to be attracted to the positive charge and thereby causing the pathogen to ingest the biocidally effective mixture, wherein the positive charge in combination with the food component facilitates entry of the biocidally effective mixture into a digestive canal of the pathogen, thereby effectively suffocating the pathogen when the wax blocks an air or digestive action pathway of the pathogen.
